(19) United States
(12) Patent Application Publication
(10) Pub. No.: US 2019/0233502 A1
(43) Pub. Date: Aug. 1, 2019

(54) PNEUMOCOCCAL INHIBITORY FACTOR COMPOSITIONS AND METHODS OF USE THEREOF

(71) Applicants: Martha A. ALEXANDER-MILLER, Clemmons, NC (US); Wake Forest University Health Sciences, Winston-Salem, NC (US)

(72) Inventor: Martha A Alexander-Miller, Clemmons, NC (US)

(21) Appl. No.: 15/779,472

(22) PCT Filed: Nov. 23, 2016
(86) PCT No.: PCT/US16/63558

§ 371 Date: May 25, 2018

Related U.S. Application Data
(60) Provisional application No. 62/260,235, filed on Nov. 25, 2015.

(51) Int. Cl. C07K 14/81; C07K 14/315; A61P 37/06; C07K 16/24; A61P 31/04; C07K 19/00; A61K 47/64; A61K 47/68

(52) U.S. Cl. CPC C07K 14/8103; C07K 14/315; A61P 37/06; C07K 16/249; A61P 31/04; C12Y 304/11022; C07K 16/246; C07K 19/00; A61K 47/642; A61K 47/646; A61K 47/6815; C07K 16/245

(57) ABSTRACT

An isolated or recombinant aminopeptidase N (pepN) polypeptide having inflammatory cytokine production and cytolysis inhibiting activity is provided. A method of inhibiting inflammatory cytokine production and cytolysis in a subject in need thereof is also provided, comprising administering to the subject an inflammatory cytokine production and cytolysis inhibiting amount of the isolated or recombinant pneumococcal pepN polypeptide. A method of treating a disease, disorder, or condition characterized by or associated with undesirable inflammatory cytokine production and cytolysis in a subject in need thereof is also provided, comprising administering to the subject a therapeutically effective amount of an isolated or recombinant pepN polypeptide. A method of treating pneumococcal infection in a subject in need thereof is also provided comprising administering to the subject a therapeutically effective amount of an anti-pepN polypeptide inhibitory agent.

### FIELD OF THE INVENTION

The invention relates to the fields of immunology and medicine, and provides pneumococcal inhibitory factor compositions and methods of use thereof.

### BACKGROUND

Infection with pneumococcus can result in severe disease. The World Health Organization (WHO) estimates that more than 1.6 million people die every year from pneumococcal infections. This includes more than 800,000 children under the age of 5. Pneumococcal meningitis is the most severe form of pneumococcal disease. In developing countries, pneumococcal meningitis kills or disables 40 to 70 percent of children who contract it (Baraff et al. (1993) Pediatr. Infect. Dis. J. 12: 389-394). Increasing rates of drug-resistant pneumococcal infections threaten the effectiveness of antibiotic treatment (Klugman (2002) Eur. Respir. J. Suppl. 36: 3s-8s; Dagan (2000) Pediatr. Infect. Dis. J. 19: 378-382). Pneumococcus is often carried in the nasopharynx, especially in children (Bogaert et al. (2004) Lancet Infect. Dis. 4: 144-154). Carriage is critical in transmission and disease and often precedes lower respiratory tract infection.

Mucosal antibody and CD4⁺ T cells play important roles in protection from pneumococcus. CD4⁺ T cells contribute through both T dependent antibody production and direct CD4⁺ T cell effector function (Malley et al. (2005) Proc. Natl. Acad. Sci. U.S.A. 102: 4848-4853; Basset et al. (2007) Infect. Immun. 75: 5460-5464; Lu et al. (2008) PLoS Pathog. 4: e1000159). In addition, in mouse models Th17 cells are essential for protection against carriage (Zhang et al. (2009) J. Clin. Invest. 119: 1899-1909; Trzcinski et al. (2008) Infect. Immun. 76: 2678-2684). The importance of CD4⁺ T cells has been validated in humans as studies suggest low CD4⁺ T cell immunity is associated with carriage in children (Zhang et al. (2007) J. Infect. Dis. 195: 1194-1202). In addition, an elegant study from Wright et al showed that experimental infection in humans resulted in a greater than 17-fold increase in IL-17 producing CD4⁺ T cells in the BAL (Wright et al. (2013) PLoS Pathog. 9: e1003274). In this model, IL-17 was shown to increase pneumococci uptake by alveolar macrophages. Thus T cells are a critical component of the control of pneumococcus infection.

Given the importance of T cells in the control of this pathogen, the ability to negatively regulate T cell function would be an effective mechanism to evade clearance. Using a mechanically disrupted preparation of Spn, a surprising discovery was made that a component of Spn can inhibit function in effector T cells. Use of disrupted bacteria mimics the autolysis that occurs during infection allowing tissues to be exposed to internal bacterial components. A novel protein, aminopeptidase N (pepN), has now been identified which reproduces the shut-off of function in effector cells, and inhibits inflammatory cytokine production and cytolysis in a dose dependent manner.

Accordingly, pepN finds use in compositions and methods to treat inflammatory cytokine-mediated diseases and disorders, while anti-pepN inhibitory agents find use in compositions and methods to treat pneumococcal infection.

### SUMMARY

In some aspects, the presently disclosed subject matter provides an isolated or recombinant aminopeptidase N (pepN) polypeptide, wherein the isolated or recombinant pepN polypeptide comprises the amino acid sequence of SEQ ID NO:1 or a functional variant thereof, wherein the functional variant thereof has inflammatory cytokine production and cytolysis inhibiting activity and comprises an amino acid sequence at least 80% identical to the amino acid sequence of SEQ ID NO:1. In another aspect, the isolated or recombinant pepN polypeptide is fused to a heterologous polypeptide, particularly wherein the heterologous polypeptide is an epitope tag. In another aspect, the functional variant is a functional fragment of an amino acid sequence at least 80% identical to the amino acid sequence of SEQ ID NO:1. In another aspect, the presently disclosed subject matter provides a composition comprising any of the isolated or recombinant pepN polypeptides described above in a pharmaceutically acceptable carrier.

In other aspects, the presently disclosed subject matter provides a method of inhibiting inflammatory cytokine production and cytolysis in a subject in need thereof, the method comprising administering to the subject an inflammatory cytokine production and cytolysis inhibiting amount of an isolated or recombinant pepN polypeptide, wherein the isolated or recombinant pepN polypeptide comprises the amino acid sequence of SEQ ID NO:1 or a functional variant thereof, wherein the functional variant thereof has inflammatory cytokine production and cytolysis inhibiting activity and comprises an amino acid sequence at least 80% identical to the amino acid sequence of SEQ ID NO:1. In another aspect, the isolated or recombinant pepN polypeptide is fused to a heterologous polypeptide, particularly wherein the heterologous polypeptide is an epitope tag. In another aspect, the functional variant is a functional fragment of an amino acid sequence at least 80% identical to the amino acid sequence of SEQ ID NO:1. In another aspect, any of the isolated or recombinant pepN polypeptides described above are in a pharmaceutically acceptable carrier. In another aspect, the inflammatory cytokine is selected from the group consisting of Interleukin 1 (IL-1), Interleukin 2 (IL-2), Interleukin 5 (IL-5), Interleukin 6 (IL-6), Interleukin 8 (IL-8), Tumor Necrosis Factor alpha (TNFα), and Interferon gamma (IFNγ). In another aspect, the subject is a human.

In other aspects, the presently disclosed subject matter provides a method of treating a disease, disorder, or condition characterized by or associated with undesirable inflammatory cytokine production and cytolysis in a subject in need thereof, the method comprising administering to the subject a therapeutically effective amount of an isolated or recombinant pepN polypeptide, wherein the isolated or recombinant pepN polypeptide comprises the amino acid sequence of SEQ ID NO:1 or a functional variant thereof, wherein the functional variant thereof has inflammatory cytokine production and cytolysis inhibiting activity and comprises an amino acid sequence at least 80% identical to the amino acid sequence of SEQ ID NO:1. In another aspect, the disease, disorder, or condition is a disease, disorder, or condition of the lungs, joints, eyes, bowel, skin, or heart. In another aspect, the disease, disorder, or condition is selected from the group consisting of asthma, adult respiratory distress syndrome, bronchitis, bronchiectasis, bronchiolitis obliterans, diffuse panbronchiolitis, cystic fibrosis, rheumatoid arthritis, rheumatoid spondylitis, osteoarthritis, osteomyelitis, sinusitis, nasal polyps, gouty arthritis, uveitis, conjunctivitis, inflammatory bowel conditions, Crohn's disease, ulcerative colitis, distal proctitis, acne, psoriasis, eczema, dermatitis, coronary infarct damage, coronary artery disease, chronic inflammation, endotoxin shock, and smooth muscle proliferation disorders. In another aspect, the isolated or recombinant pepN polypeptide is fused to a heterologous polypeptide, particularly wherein the heterologous polypeptide is an epitope tag. In another aspect, the functional variant is a functional fragment of an amino acid sequence at least 80% identical to the amino acid sequence of SEQ ID NO:1. In another aspect, any of the isolated or recombinant pepN polypeptides described above are in a pharmaceutically acceptable carrier. In another aspect, the inflammatory cytokine is selected from the group consisting of IL-1, IL-2, IL-5, IL-6, IL-8, TNFα, and IFNγ. In another aspect, the subject is a human.

In other aspects, the presently disclosed subject matter provides a method of treating pneumococcal infection in a subject in need thereof, the method comprising administering to the subject a therapeutically effective amount of an anti-pepN polypeptide inhibitory agent, wherein the anti-pepN polypeptide inhibitory agent is an anti-pepN polypeptide antibody or antigen-binding fragment thereof, a small molecule pepN polypeptide inhibitor, an RNA or DNA aptamer that binds or physically interacts with a pepN polypeptide, a soluble pepN polypeptide receptor, a pepN polypeptide specific antisense molecule, or a pepN polypeptide specific siRNA molecule. In another aspect, the pepN polypeptide is an isolated or recombinant pepN polypeptide comprising the amino acid sequence of SEQ ID NO:1 or a functional variant thereof, wherein the functional variant thereof has inflammatory cytokine production and cytolysis inhibiting activity and comprises an amino acid sequence at least 80% identical to the amino acid sequence of SEQ ID NO:1. In another aspect, the functional variant is a functional fragment of an amino acid sequence at least 80% identical to the amino acid sequence of SEQ ID NO:1. In another aspect, the subject is a human.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

Having thus described the presently disclosed subject matter in general terms, reference will now be made to the accompanying Drawings, which are not necessarily drawn to scale, and wherein:

FIG. 1 shows that the addition of Spn components to CD8⁺ lung effector cells inhibits their ability to produce IFNγ in response to peptide stimulation;

FIG. 2 shows that CD4⁺ effectors are inhibited by Spn components (two concentrations of lysate are shown);

FIG. 3 shows that effector cells stimulated in the presence of Spn lysate have a reduced level of IFNγ mRNA;

FIG. 4 shows that treatment with lysate from a variety of both clinical and laboratory strains encompassing multiple serotypes can inhibit cytokine production (ND=not done);

FIG. 5 shows: A) Expression of pepN-His in E. coli; and B) Treatment with EF3030 pepN-His inhibits cytokine production by T effector cells; and

FIG. 6 shows that effector cells stimulated in the presence of Spn pepN-His are greatly inhibited for IFNγ mRNA production.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

Pneumococcal infection is the second most common cause of fatal bacterial infection across the globe (Rothberg et al. (2008) Am. J. Med. 121: 258-264). The World Health Organization (WHO) estimates that worldwide approximately 14.5 million episodes of serious pneumococcal disease occur, resulting in about 826,000 deaths in children aged 1-59 months. Streptococcus pneumoniae (Spn) has a number of virulence factors that contribute to its ability to cause disease including polysaccharide capsule, pneumolysin and pneumococcal surface proteins A-C (Feldman & Anderson (2014) F1000 Prime Rep 6: 82). These factors promote adherence as well as escape from host defenses. With regard to the latter, capsular polysaccharide effectively prevents phagocytosis (Steel et al. (2013) Mediators Inflamm. 2013: 490346), while pneumolysin can kill immune cells (Littmann et al. (2009) EMBO Mol. Med. 1: 211-222).

The presently disclosed subject matter relates to the identification of a novel immunoregulatory property of Spn. As described in the Examples below, exposure of murine effector cells to a soluble fraction generated from mechanically disrupted Spn resulted in effective inhibition of cytokine production. Use of disrupted bacteria mimicked the autolysis that occurs during infection which results in exposure of tissue to internal bacterial components. The inhibition observed was a highly novel and unexpected property of pneumococcus. Importantly, this effect was not restricted to the EF3030 strain of pneumococcus. An array of clinical and laboratory isolates encompassing multiple serotypes were tested, finding that all could inhibit T effector cell function.

In characterizing the inhibitory factor, it was found to be heat labile and protease sensitive. Through a series of fractionation and sequencing approaches, candidate molecules were identified. Two candidates were cloned, engineered to express a His tag, and expressed in E. coli, allowing for efficient isolation for further testing. As described in the Examples below, data showed that one of the candidates, aminopeptidase N (pepN), could reproduce the T cell inhibitory effect observed with disrupted Spn.

The pepN protein from Streptococcus pneumoniae has not been previously isolated or studied. The ability of pepN to actively regulate function in effector cells presents a new mechanism for immune regulation by this clinically important bacterium. Specifically, pepN reproduces the shut-off of function in T effector cells, and inhibits inflammatory cytokine production and cytolysis in a dose dependent manner. Accordingly, pepN finds use in compositions and methods to treat inflammatory cytokine-mediated diseases and disorders, while anti-pepN inhibitory agents find use in compositions and methods to treat pneumococcal infection.

I. Aminopeptidase N (pepN) Polypeptides and Related Compositions

In some aspects, the presently disclosed subject matter provides an isolated or recombinant aminopeptidase N (pepN) polypeptide, wherein the isolated or recombinant pepN polypeptide comprises the amino acid sequence of SEQ ID NO:1 or a functional variant thereof, wherein the functional variant thereof has inflammatory cytokine production and cytolysis inhibiting activity and comprises an amino acid sequence at least 40%, 45%, 50%, 55%, 60%, 65%, 70%, 75%, 80%, 85%, 90%, 91%, 92%, 93%, 94%, 95%, 96%, 97%, 98%, 99% or more identical to the amino acid sequence of SEQ ID NO:1. In another aspect, the functional variant is a functional fragment of an amino acid sequence at least 40%, 45%, 50%, 55%, 60%, 65%, 70%, 75%, 80%, 85%, 90%, 91%, 92%, 93%, 94%, 95%, 96%, 97%, 98%, 99% or more identical to the amino acid sequence of SEQ ID NO:1. The amino acid sequence of SEQ ID NO:1 is provided in Table 1.

TABLE 1
Amino Acid Sequence of Aminopeptidase N (pepN)
Polypeptide

MQAVEHFIKQFVPEHYDLFLDLSRETKTFSGKVTITGQAQSDRISLHQKD
LEITSVEVAGQARPFTVDHDNEALHIELAEAGQVELVLAFSGKITDNMTG
IYPSYYTVDGVKKEVLSTQFESHFAREAFPCVDEPEAKATFDLALRFDQA
AGELALSNMPEIDVDNRKETGIWKFETTPRMSSYLLAFVAGDLQGVTAKT
KNGALVGVYSTKAHPLSNLDFSLDIAVRSIEFYEDYYGVKYPIPQSLHIA
LPDFSAGAMENWGLVTYREVYLVVDENSTFASRQQVALVVAAALAAQWFG
NLVTMKWWDDLWLNESFANMMEYVCVDTIEPSWNIFEDFQTGGVPLALER
DATDGVQSVHVEVKHPDEINTLFDGAIVYAKGSRLMHMLRRWLGDADFAK
GLHAYFEKHQYSNTIGSDLWDALGQASGRDVAAFMDSWLEQPGYPVLTVK
VENDVLKISQKQFFIGENEDKNRLWVVPLNSNWKGLPDTLETESIEIPGY
AALLAENEGALRLNTENTAHYITDYQGDLLEAVLAELETLDNTSKLQIVQ
ERRLLAEAGHISYADLLPVLDKLAKEESYLVVSAVSQVISALERFIDEGT
DAETAFKGLVAKLARHNYDRLGFEAKDGESDEDELVRQLAVSMMIRSNDA
EASQVASQIFATHKENLAGLPAAIRSQVLINEMKHHETKDLLALYLDTYT
HATDAVFKRQLAAALAYSTDADNIQNLITSWKDKFVVKPQDLSAWYYQFL
AHQATQKTAWSWARENWAWIKAALGGDMSFDSFVILPAHVFKTQQRLAEY
KEFFEPQLSDLALSRNIGMGIKEIAARVDLISREKAAVEAVVLQYGNA
(SEQ ID NO: 1)

The terms “polypeptide,” “peptide,” and “protein” are used interchangeably herein to refer to a polymer of amino acid residues. The terms apply to amino acid polymers in which one or more amino acid residues is an artificial chemical analogue of a corresponding naturally occurring amino acid, as well as to naturally occurring amino acid polymers. Polypeptides of the invention can be produced either from a nucleic acid disclosed herein, or by the use of standard molecular biology techniques. For example, a truncated protein of the invention can be produced by expression of a recombinant nucleic acid of the invention in an appropriate host cell, or alternatively by a combination of ex vivo procedures, such as protease digestion and purification.

The presently disclosed subject matter encompasses isolated or substantially purified polypeptide compositions. An “isolated” or “purified” polypeptide is substantially or essentially free from components that normally accompany or interact with the polypeptide as found in its naturally occurring environment. A polypeptide that is substantially free of cellular material includes preparations of protein having less than about 30%, 20%, 10%, 5%, or 1% (by dry weight) of contaminating protein. When the polypeptide of the presently disclosed subject matter is recombinantly produced, optimally culture medium represents less than about 30%, 20%, 10%, 5%, or 1% (by dry weight) of chemical precursors or non-protein-of-interest chemicals.

Fragments and variants of the disclosed polypeptides are also encompassed by the presently disclosed subject matter. Fragments and variants of a polypeptide that retain the biological activity of the native pepN polypeptide and hence inhibit inflammatory cytokine production and cytolysis by T effector cells are referred to herein as “functional fragments” or “functional variants” of the pepN polypeptide.

“Fragment” is intended to mean a portion of the full-length sequence. As used herein, “full-length sequence” in reference to a specified polypeptide means having the entire amino acid sequence of a native sequence. “Native sequence” or “native polypeptide” is intended to mean an endogenous sequence or polypeptide, i.e., a non-engineered sequence or polypeptide found in vivo in an organism.

“Variant” is intended to mean a substantially similar sequence to a native sequence. A variant polypeptide is intended to mean a polypeptide derived from the native polypeptide by deletion or addition of one or more amino acids at one or more internal sites in the native polypeptide and/or substitution of one or more amino acids at one or more sites in the native polypeptide. Functional variants may result from, for example, genetic polymorphism or from human manipulation. Functional variants of a native pepN polypeptide of the presently disclosed subject matter will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence for the native polypeptide as determined by sequence alignment programs and parameters described elsewhere herein. A biologically active variant of a polypeptide of the presently disclosed subject matter may differ from that polypeptide by as few as 1-15 amino acid residues, as few as 1-10, such as 6-10, as few as 5, as few as 4, 3, 2, or even 1 amino acid residue.

The polypeptides of the presently disclosed subject matter may be altered in various ways including amino acid substitutions, deletions, truncations, and insertions. Methods for such manipulations are generally known in the art. For example, amino acid sequence variants and fragments of the pepN polypeptide can be prepared by mutations in DNA encoding such polypeptides. Methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of a protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

In nature, some polypeptides are produced as complex precursors which, in addition to targeting labels such as the signal peptides discussed elsewhere in this application, also contain other fragments of peptides which are removed (processed) at some point during protein maturation, resulting in a mature form of the polypeptide that is different from the primary translation product (aside from the removal of the signal peptide). “Mature protein” refers to a post-translationally processed polypeptide; i.e., one from which any pre- or propeptides present in the primary translation product have been removed. “Precursor protein” or “prepropeptide” or “preproprotein” all refer to the primary product of translation of mRNA; i.e., with pre- and propeptides still present. Pre- and propeptides may include, but are not limited to, intracellular localization signals. “Pre” in this nomenclature generally refers to the signal peptide. The form of the translation product with only the signal peptide removed but no further processing yet is called a “propeptide” or “proprotein.” The fragments or segments to be removed may themselves also be referred to as “propeptides.” A proprotein or propeptide thus has had the signal peptide removed, but contains propeptides (here referring to propeptide segments) and the portions that will make up the mature protein. The skilled artisan is able to determine, depending on the species in which the proteins are being expressed and the desired intracellular location, if higher expression levels might be obtained by using a gene construct encoding just the mature form of the protein, the mature form with a signal peptide, or the proprotein (i.e., a form including propeptides) with a signal peptide.

The deletions, insertions, and substitutions of the protein sequences encompassed herein are not expected to produce radical changes in the characteristics of the protein. However, when it is difficult to predict the exact effect of the substitution, deletion, or insertion in advance of doing so, one skilled in the art will appreciate that the effect will be evaluated by routine screening assays. That is, the activity can be evaluated by assays that measure inflammatory cytokine production and cytolysis, such as those described in the Examples below.

The following terms are used to describe the sequence relationships between two or more polypeptides: (a) “reference sequence”; (b) “comparison window”; (c) “sequence identity”; and, (d) “percentage of sequence identity.”

As used herein, “reference sequence” is a defined sequence used as a basis for sequence comparison. A reference sequence may be a subset or the entirety of a specified sequence; for example, as a segment of a full-length amino acid sequence or the complete amino acid sequence.

As used herein, “comparison window” makes reference to a contiguous and specified segment of a polypeptide sequence, wherein the polypeptide sequence in the comparison window may comprise additions or deletions (i.e., gaps) compared to the reference sequence (which does not comprise additions or deletions) for optimal alignment of the two polypeptides.

As used herein, “sequence identity” or “identity” in the context of two amino acid sequences makes reference to the residues in the two sequences that are the same when aligned for maximum correspondence over a specified comparison window. When percentage of sequence identity is used in reference to polypeptides it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

As used herein, “percentage of sequence identity” means the value determined by comparing two optimally aligned sequences over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the window of comparison, and multiplying the result by 100 to yield the percentage of sequence identity.

In another aspect of the presently disclosed subject matter, the isolated or recombinant pepN polypeptide is fused to a heterologous polypeptide, particularly wherein the heterologous polypeptide is an epitope tag. The term “epitope tagged” when used herein refers to a chimeric polypeptide comprising a pepN polypeptide or functional variant thereof fused to a “tag polypeptide.” The tag polypeptide has enough residues to provide an epitope against which an antibody can be made, yet is short enough such that it does not interfere with activity of the polypeptide to which it is fused. The tag polypeptide preferably also is fairly unique so that the antibody does not substantially cross-react with other epitopes. Suitable tag polypeptides generally have at least six amino acid residues and usually between about 8 and 50 amino acid residues (preferably, between about 10 and 20 amino acid residues). The epitope tag is generally placed at the amino- or carboxyl-terminus of the pepN polypeptide or functional variant thereof. The presence of such epitope-tagged forms of the pepN polypeptides or functional variants thereof can be detected using an antibody against the tag polypeptide. Also, provision of the epitope tag enables the pepN polypeptides or functional variants thereof to be readily purified by affinity purification using an anti-tag antibody or another type of affinity matrix that binds to the epitope tag. Various tag polypeptides and their respective antibodies are well known in the art. Examples include poly-histidine (poly-his) or poly-histidine-glycine (poly-his-gly) tags; the flu HA tag polypeptide and its antibody 12CA5; the c-myc tag and the 8F9, 3C7, 6E10, G4, B7 and 9E10 antibodies thereto; and the Herpes Simplex virus glycoprotein D (gD) tag and its antibody. Other tag polypeptides include the Flag-peptide; the KT3 epitope peptide; an a-tubulin epitope peptide; and the T7 gene 10 protein peptide tag.

In another aspect of the presently disclosed subject matter, a composition is provided comprising any of the isolated or recombinant pepN polypeptides described above in a pharmaceutically acceptable carrier. The phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” as used herein means a pharmaceutically acceptable material, composition or vehicle, such as a liquid or solid filler, diluent, excipient, solvent, media, encapsulating material, manufacturing aid (e.g., lubricant, talc magnesium, calcium or zinc stearate, or steric acid), or solvent encapsulating material, involved in maintaining the stability, solubility, or activity of, a pepN polypeptide. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) excipients, such as cocoa butter and suppository waxes; (8) oils, such as peanut oil, cottonseed oil, safflower oil, sesame oil, olive oil, corn oil and soybean oil; (9) glycols, such as propylene glycol; (10) polyols, such as glycerin, sorbitol, mannitol and polyethylene glycol (PEG); (11) esters, such as ethyl oleate and ethyl laurate; (12) agar; (13) buffering agents, such as magnesium hydroxide and aluminum hydroxide; (14) alginic acid; (15) pyrogen-free water; (16) isotonic saline; (17) Ringer's solution; (19) pH buffered solutions; (20) polyesters, polycarbonates and/or polyanhydrides; (21) bulking agents, such as polypeptides and amino acids (22) serum components, such as serum albumin, HDL and LDL; (23) C2-C12 alcohols, such as ethanol; and (24) other non-toxic compatible substances employed in pharmaceutical formulations. Release agents, coating agents, preservatives, and antioxidants can also be present in the formulation. The terms such as “excipient,” “carrier,” “pharmaceutically acceptable carrier,” or the like are used interchangeably herein.

### II. Methods of Inhibiting Inflammatory Cytokine Production and Cytolysis and Related Treatment Methods

In other aspects, the presently disclosed subject matter provides a method of inhibiting inflammatory cytokine production and cytolysis in a subject in need thereof, the method comprising administering to the subject an inflammatory cytokine production and cytolysis inhibiting amount of an isolated or recombinant pepN polypeptide, wherein the isolated or recombinant pepN polypeptide comprises the amino acid sequence of SEQ ID NO:1 or a functional variant thereof, wherein the functional variant thereof has inflammatory cytokine production and cytolysis inhibiting activity and comprises an amino acid sequence at least 40%, 45%, 50%, 55%, 60%, 65%, 70%, 75%, 80%, 85%, 90%, 91%, 92%, 93%, 94%, 95%, 96%, 97%, 98%, 99% or more identical to the amino acid sequence of SEQ ID NO:1. In another aspect, the isolated or recombinant pepN polypeptide is fused to a heterologous polypeptide, particularly wherein the heterologous polypeptide is an epitope tag. In another aspect, the functional variant is a functional fragment of an amino acid sequence at least 40%, 45%, 50%, 55%, 60%, 65%, 70%, 75%, 80%, 85%, 90%, 91%, 92%, 93%, 94%, 95%, 96%, 97%, 98%, 99% or more identical to the amino acid sequence of SEQ ID NO:1.

Inflammatory cytokines are cytokines that promote inflammation. “Cytokine” is a general term for a variety of physically active substances responsible for intercellular signal transductions. Cytokines form a group of substances involved in regulating various biological functions by participating in intracellular signal transduction via specific receptors. Examples of biological functions in which cytokines are involved include biological defense and immune responses. Of these, inflammation is deeply associated with cytokines. Among cytokines, Interleukin 1 (IL-1), Interleukin 2 (IL-2), Interleukin 5 (IL-5), Interleukin 6 (IL-6), Interleukin 8 (IL-8), Tumor Necrosis Factor alpha (TNFα), and Interferon gamma (IFNγ) and the like act to promote inflammation, and therefore, are classified as inflammatory cytokines. In addition, cytokines having an activity to regulate the production of these inflammatory cytokines are referred to as anti-inflammatory cytokines (Moore et al. (2001) Annu. Rev. Immunol. 19:683-765; Mosmann (1994) Adv. Immunol. 56:1-26). These inflammatory cytokines and anti-inflammatory cytokines are known to appropriately regulate their mutual production level and activities (Mosmann (1994) Adv. Immunol. 56:1-26).

One skilled in the art will appreciate that “an inflammatory cytokine production and cytolysis inhibiting amount” of an isolated or recombinant pepN polypeptide may be evaluated by routine screening assays. That is, the activity can be evaluated by assays that measure inflammatory cytokine production and cytolysis, such as those described in the Examples below.

In other aspects, the presently disclosed subject matter provides a method of treating a disease, disorder, or condition characterized by or associated with undesirable inflammatory cytokine production and cytolysis in a subject in need thereof, the method comprising administering to the subject a therapeutically effective amount of an isolated or recombinant pepN polypeptide, wherein the isolated or recombinant pepN polypeptide comprises the amino acid sequence of SEQ ID NO:1 or a functional variant thereof, wherein the functional variant thereof has inflammatory cytokine production and cytolysis inhibiting activity and comprises an amino acid sequence at least 40%, 45%, 50%, 55%, 60%, 65%, 70%, 75%, 80%, 85%, 90%, 91%, 92%, 93%, 94%, 95%, 96%, 97%, 98%, 99% or more identical to the amino acid sequence of SEQ ID NO:1. In another aspect, the functional variant is a functional fragment of an amino acid sequence at least 40%, 45%, 50%, 55%, 60%, 65%, 70%, 75%, 80%, 85%, 90%, 91%, 92%, 93%, 94%, 95%, 96%, 97%, 98%, 99% or more identical to the amino acid sequence of SEQ ID NO:1.

In particular aspects, the disease, disorder, or condition characterized by or associated with undesirable inflammatory cytokine production and cytolysis is a disease, disorder, or condition of the lungs, joints, eyes, bowel, skin, or heart. In other particular aspects, the disease, disorder, or condition characterized by or associated with undesirable inflammatory cytokine production and cytolysis is selected from the group consisting of asthma, adult respiratory distress syndrome, bronchitis, bronchiectasis, bronchiolitis obliterans, diffuse panbronchiolitis, cystic fibrosis, rheumatoid arthritis, rheumatoid spondylitis, osteoarthritis, osteomyelitis, sinusitis, nasal polyps, gouty arthritis, uveitis, conjunctivitis, inflammatory bowel conditions, Crohn's disease, ulcerative colitis, distal proctitis, acne, psoriasis, eczema, dermatitis, coronary infarct damage, coronary artery disease, chronic inflammation, endotoxin shock, and smooth muscle proliferation disorders. In another aspect, the inflammatory cytokine is selected from the group consisting of IL-1, IL-2, IL-5, IL-6, IL-8, TNFα, and IFNγ.

### III. Methods of Treating Pneumococcal Infection

In other aspects, the presently disclosed subject matter provides a method of treating pneumococcal infection in a subject in need thereof, the method comprising administering to the subject a therapeutically effective amount of an anti-pepN polypeptide inhibitory agent, wherein the anti-pepN polypeptide inhibitory agent is an anti-pepN polypeptide antibody or antigen-binding fragment thereof, a small molecule pepN polypeptide inhibitor, an RNA or DNA aptamer that binds or physically interacts with a pepN polypeptide, a soluble pepN polypeptide receptor, a pepN polypeptide specific antisense molecule, or a pepN polypeptide specific siRNA molecule. In another aspect, the pepN polypeptide is an isolated or recombinant pepN polypeptide comprising the amino acid sequence of SEQ ID NO:1 or a functional variant thereof, wherein the functional variant thereof has inflammatory cytokine production and cytolysis inhibiting activity and comprises an amino acid sequence at least 40%, 45%, 50%, 55%, 60%, 65%, 70%, 75%, 80%, 85%, 90%, 91%, 92%, 93%, 94%, 95%, 96%, 97%, 98%, 99% or more identical to the amino acid sequence of SEQ ID NO:1. In another aspect, the functional variant is a functional fragment of an amino acid sequence at least 40%, 45%, 50%, 55%, 60%, 65%, 70%, 75%, 80%, 85%, 90%, 91%, 92%, 93%, 94%, 95%, 96%, 97%, 98%, 99% or more identical to the amino acid sequence of SEQ ID NO:1.

Streptococcus pneumoniae (Spn), also named pneumococcus or Diplococcus pneumonia, is a Gram-positive bacterial strain and a member of the genus Streptococcus which undergoes cellular division along a single axis therein and thus grows in chains or pairs. Spn is recognized as a major cause of pneumonia. Spn infects the normal upper respiratory tract, causing pneumonia. In fact, Spn is reported to be responsible for as much as about 70% of the bacterial pneumonia cases. Also, the organism causes many types of pneumococcal infection in various tissues other than pneumonia in the lung, including bacteremia in blood, osteomyelitis in the bone, otitis media in the ear, peritonitis in the stomach or the duodenum, pericarditis in the pericardium, and cellulitis in general wound sites. Spn is known as the most common cause of pneumonia bacterial meningitis in infants and children. In addition, Spn may be fatal to chronic patients with heart failure or diabetes and may propagate between individuals through the saliva or mucous discharges.

### IV. General Definitions

The term “administering” as used herein refers to contacting at least a cell with an isolated or recombinant pepN polypeptide or an anti-pepN polypeptide inhibitory agent as defined herein. This term includes administration of the presently disclosed an isolated or recombinant pepN polypeptide or an anti-pepN polypeptide inhibitory agent to a subject in which the cell is present, as well as introducing the presently disclosed isolated or recombinant pepN polypeptide or anti-pepN polypeptide inhibitory agent into a medium in which a cell is cultured.

The subject treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing disease, disorder, condition or the prophylactic treatment for preventing the onset of a disease, disorder, or condition or an animal subject for medical, veterinary purposes, or developmental purposes. Suitable animal subjects include mammals including, but not limited to, primates, e.g., humans, monkeys, apes, gibbons, chimpanzees, orangutans, macaques and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, guinea pigs, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a disease, disorder, or condition. Thus, the terms “subject” and “patient” are used interchangeably herein. Subjects also include animal disease models (e.g., rats or mice used in experiments and the like).

As used herein, the terms “treat,” treating,” “treatment,” and the like, are meant to decrease, suppress, attenuate, diminish, arrest, the underlying cause of a disease, disorder, or condition, or to stabilize the development or progression of a disease, disorder, condition, and/or symptoms associated therewith. The terms “treat,” “treating,” “treatment,” and the like, as used herein can refer to curative therapy, prophylactic therapy, and preventative therapy. Treatment according to the presently disclosed methods can result in complete relief or cure from a disease, disorder, or condition, or partial amelioration of one or more symptoms of the disease, disease, or condition, and can be temporary or permanent. The term “treatment” also is intended to encompass prophylaxis, therapy and cure.

As used herein, the terms “prevent,” “preventing,” “prevention,” “prophylactic treatment” and the like refer to reducing the probability of developing a disease, disorder, or condition in a subject, who does not have, but is at risk of or susceptible to developing a disease, disorder, or condition. Thus, in some embodiments, an agent and/or polysaccharide antigen can be administered prophylactically to prevent the onset of a disease, disorder, or condition, or to prevent the recurrence of a disease, disorder, or condition.

The term “effective amount,” as in “a therapeutically effective amount,” of a therapeutic agent refers to the amount of the agent necessary to elicit the desired biological response. As will be appreciated by those of ordinary skill in this art, the effective amount of an agent may vary depending on such factors as the desired biological endpoint, the agent to be delivered, the composition of the pharmaceutical composition, the target tissue or cell, and the like. More particularly, the term “effective amount” refers to an amount sufficient to produce the desired effect, e.g., to reduce or ameliorate the severity, duration, progression, or onset of a disease, disorder, or condition, or one or more symptoms thereof prevent the advancement of a disease, disorder, or condition, cause the regression of a disease, disorder, or condition; prevent the recurrence, development, onset or progression of a symptom associated with a disease, disorder, or condition, or enhance or improve the prophylactic or therapeutic effect(s) of another therapy.

In some aspects, an effective amount of an isolated or recombinant pepN polypeptide is an amount that inhibits inflammatory cytokine production and cytolysis in a subject in need thereof. In particular aspects, administration of an isolated or recombinant pepN polypeptide as described herein results in at least about a 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, or 100-fold decrease in inflammatory cytokine production and cytolysis in a subject in need thereof. In other particular aspects, administration of an isolated or recombinant pepN polypeptide as described herein results in at least about a 10%, 15%, 20%, 25%, 30%, 35%, 40%, 45%, 50%, 55%, 60%, 65%, 70%, 75%, 80%, 85%, 90%, 95%, or even 100% decrease in inflammatory cytokine production and cytolysis in a subject in need thereof.

In some aspects, an effective amount of an anti-pepN polypeptide inhibitory agent is an amount that results in at least about a 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, or 100-fold decrease in one or more (e.g., 1, 2, 3, 4, 5, 6, 7, 8. 9, or 10) symptoms of a disease, disorder, or condition associated with an Spn viral infection or the likelihood of developing a disease, disorder, or condition associated with an Spn viral infection as compared to a subject that is not administered an anti-pepN polypeptide inhibitory agent as described herein. In other aspects, an effective amount of an anti-pepN polypeptide inhibitory agent is an amount that results in at least about a 10%, 15%, 20%, 25%, 30%, 35%, 40%, 45%, 50%, 55%, 60%, 65%, 70%, 75%, 80%, 85%, 90%, 95%, or even 100% decrease in one or more (e.g., 1, 2, 3, 4, 5, 6, 7, 8. 9, or 10) symptoms of a disease, disorder, or condition associated with an Spn viral infection or the likelihood of developing a disease, disorder, or condition associated with an Spn viral infection as compared to a subject that is not administered an anti-pepN polypeptide inhibitory agent as described herein.

By the term “decrease” is meant to inhibit, suppress, attenuate, diminish, arrest, or stabilize a symptom of a disease, disorder, or condition. It will be appreciated that, although not precluded, treating a disease, disorder or condition does not require that the disease, disorder, condition or symptoms associated therewith be completely eliminated.

In another aspect, the isolated or recombinant pepN polypeptide or anti-pepN polypeptide inhibitory agent can be administered to the subject by any suitable route of administration, including orally, nasally, transmucosally, ocularly, rectally, intravaginally, parenterally, including intramuscular, subcutaneous, intramedullary injections, as well as intrathecal, direct intraventricular, intravenous, intra-articular, intra-sternal, intra-synovial, intra-hepatic, intralesional, intracranial, intraperitoneal, intranasal, or intraocular injections, intracisternally, topically, as by powders, ointments or drops (including eyedrops), including buccally and sublingually, transdermally, through an inhalation spray, or other modes of delivery known in the art.

Dosages of the therapeutic agents used in the presently disclosed subject matter must ultimately be set by an attending physician. Accordingly, the dosage range for administration will be adjusted by the physician as necessary. It will be appreciated that an amount of an agent required for achieving the desired biological response may be different from the amount of agent effective for another purpose. Actual dosage levels of the agents described herein can be varied so as to obtain an amount of the agent that is effective to achieve the desired therapeutic response for a particular subject, composition, route of administration, and disease, disorder, or condition without being toxic to the subject. The selected dosage level will depend on a variety of factors including the activity of the particular agent employed, or salt thereof, the route of administration, the time of administration, the rate of excretion of the particular agent being employed, the duration of the treatment, other drugs, agents and/or materials used in combination with the particular agent employed, the age, sex, weight, condition, general health and prior medical history of the patient being treated, and like factors well known in the medical arts.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

Very little is known regarding the ability of pneumococcal products to directly regulate T cell function. The data presented here reveal a previously unknown and unexpected function for the pneumococcal protein pepN. Bacterial aminopeptidases have not been reported to modulate immune cells function and thus this observation leads to a fundamental shift in our understanding of pepN as well as host-pathogen interaction and the potential for immunoregulation by pneumococcus.

### Example 1

### Exposure to Bacterial Products Results in Inhibition of Cytokine Production at Low Concentrations and Death at Higher Concentrations

The presence of Spn infection in vivo can be associated with impaired T cell function (Blevins et al. (2014) J. Immunol. 193: 5076-5087). To test the potential for bacterial products to directly regulate T cells, isolated lung effector cells from influenza virus infected BALB/c mice (d8 p.i.) were stimulated in the presence of a bacterial lysate generated from the EF3030 strain of pneumococcus. Lysate was produced by growing Spn overnight followed by mechanical disruption by multiple passages through an Emulsiflex C3 emulsifier. Disrupted bacteria were centrifuged to remove non-soluble components. Use of disrupted bacteria mimics the autolysis that occurs during infection where tissues are exposed to internal bacterial components. Cells were cultured in the presence of NP-peptide together with Spn lysate (3 μg) for 5 hours and IFNγ production in CD8⁺ T cells determined by standard ICCS.

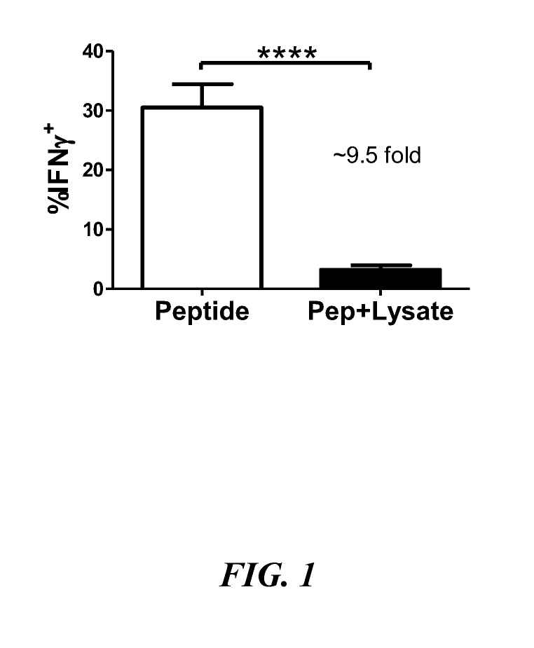

The presence of lysate resulted in a near complete inhibition of peptide-stimulated IFNγ production (FIG. 1, lung cells are shown, similar results were obtained with lymph node cells). The inhibition of function was not associated with cell death as indicated by the absence of changes in the FSC/SSC profile combined with the failure to observe an increase in 7-AAD positivity. We have extended incubation with the lysate to 16 h with no increased loss viability. Thus, there is no evidence of death as the mediator of the dysregulated cytokine production. Finally, TNFα production is similarly inhibited by the presence of Spn lysate. The ability of Spn components to inhibit effector function is not restricted to influenza-specific cells, as LCMV-specific in vivo derived effectors are also inhibited in the presence of Spn lysate.

Without being bound by any particular theory, one possibility to explain reduction in cytokine production in effectors following treatment with Spn components was alteration in the membrane proximal TCR signaling cascade. If this were the case, stimulation by addition of phorbol 12-myristate 13-acetate (PMA) and ionomycin should promote cytokine production cells cultured in the presence of lysate as these agents bypass the TCR by directly inducing PKC activation and calcium flux, respectively. PMA/ION stimulation did not increase IFNγ production relative to peptide stimulation in the NP-specific cells isolated from the lungs of influenza virus infected animals, thereby excluding membrane proximal defects in TCR signaling as the mechanism responsible for the failure to produce cytokine in effectors.

### Example 2

### CD4⁺ Effectors are Inhibited by Spn Components

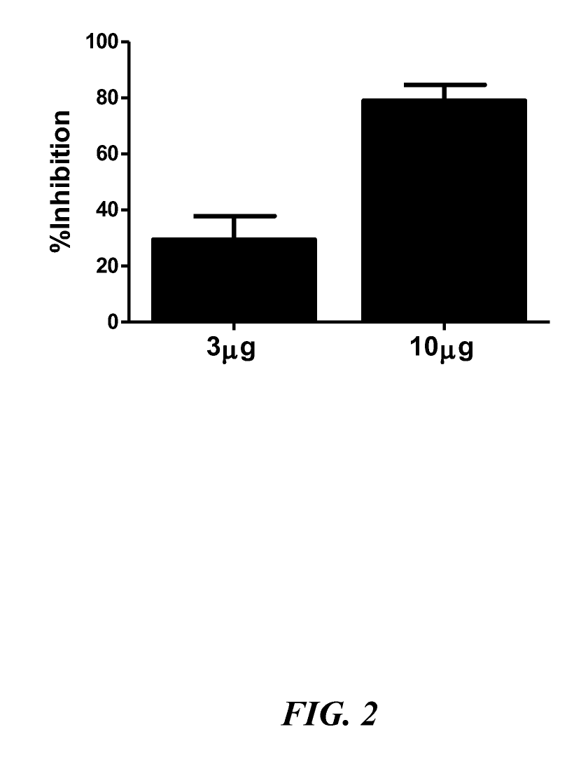

To determine whether the negative regulation associated with Spn-derived components extended to CD4⁺ effector cells, we utilized effectors generated from D011.10 TCR transgenic mice. These cells were activated in vitro by stimulation with peptide in the presence of T-stim as a source of IL-2. On day 7 following routine stimulation, cells were stimulated with peptide in the presence of Spn-derived components and IFNγ production measured by ICCS. As shown above for CD8⁺ cells, CD4⁺ effector function was inhibited by Spn (FIG. 2). Thus Spn-derived components have the potential to inhibit both CD4⁺ and CD8⁺ effectors.

### Example 3

### Inhibition is Independent of Pneumolysin

A number of studies have reported the ability of pneumococcus derived pneumolysin to negatively regulate lymphocytes (Daigneault et al. (2012) PLoS Pathog. 8: e1002814; Browne et al. (1999) Molecular & Cellular Biology 19: 8604-8615). To determine whether the functional inhibition observed with Spn-derived lysate could be attributed to pneumolysin, we employed a D39 strain that lacks this protein (PLY def) (Berry et al. (1989) Infect. Immun. 57: 2037-2042). A preliminary study confirmed the ability of D39 derived lysate to inhibit function. Functional inhibition was similar in the presence or absence of pneumolysin. Thus, a novel bacterial component(s) appeared to be responsible for the inhibition of effector cell function in our model.

### Example 4

### The Failure to Produce IFNγ is Associated with Reduced mRNA

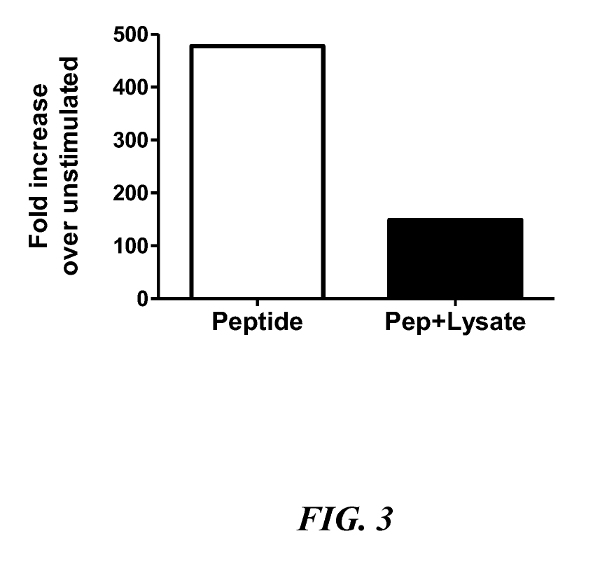

To begin to understand the regulation of cytokine production at a mechanistic level, we determined whether the failure to produce cytokine was associated with a decrease in message. For this study we utilized in vitro maintained primary influenza NP-specific CD8⁺ effector cells originally obtained from influenza virus infected BALB/c mice. Effector cells were stimulated with peptide in the presence or absence of Spn components for 5 hours. RNA was then isolated and qRT-PCR performed with primer probe sets specific for IFNγ and GAPDH. As shown in FIG. 3, a significantly reduced level of IFNγ mRNA was detected in CD8⁺ cells when Spn components were present during the stimulation. No differences were observed in GAPDH levels in lysate treated vs. non-treated cells. These data show that the lack of IFNγ production by NP-specific CD8⁺ T cells is regulated at a step prior to transcription.

### Example 5

### Both Clinical and Laboratory Strains of Spn are Capable of Inhibiting Function in CD8⁺ Effector Cells

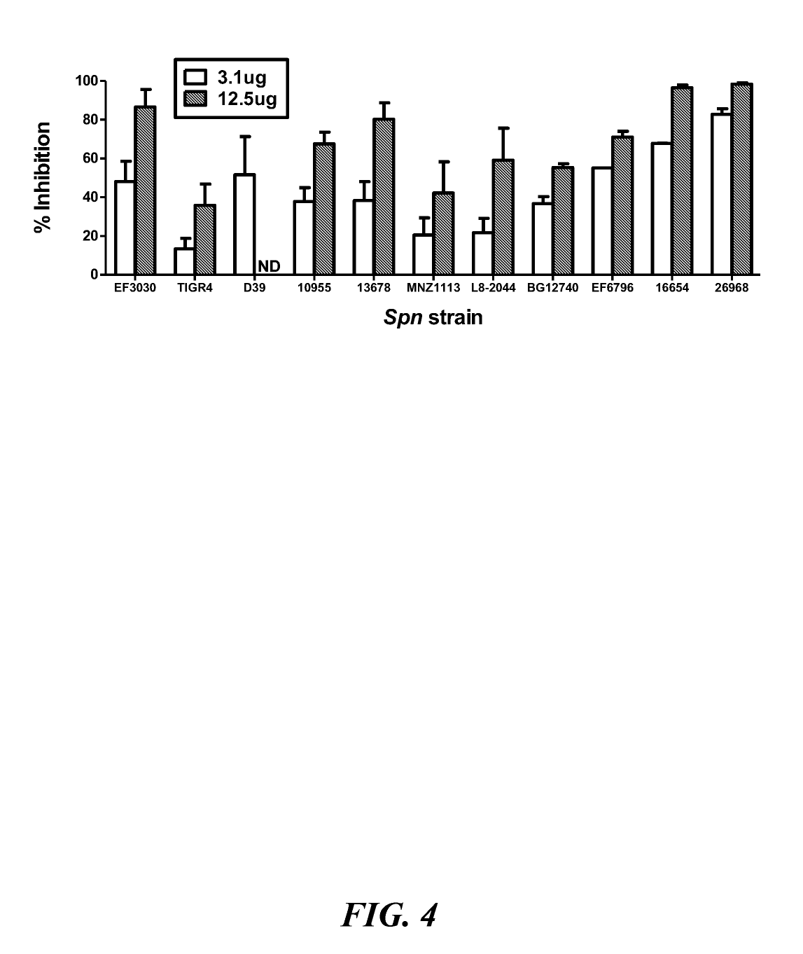

To determine whether the effects we observed were generalizable across multiple strains, we employed a number of pneumococcal strains that included laboratory (TIGR4, D39) as well as clinical isolates (MNZ1113, L8-2044, BG12740, EF6796, 16654, 26968, 10955, 13678). Lysate was added at 3 μg/well, the dose established to have strong inhibitory activity for EF3030. The data in FIG. 4 show that lysate derived from all of the strains was capable of inhibiting function in CD8⁺ effectors, although the effectiveness varied to some extent. These data show that the inhibitory component is present across pneumococcus strains.

### Example 6

### Purified EF3030 pepN-His Can Inhibit Cytokine Production by CD8⁺ Effector Cells

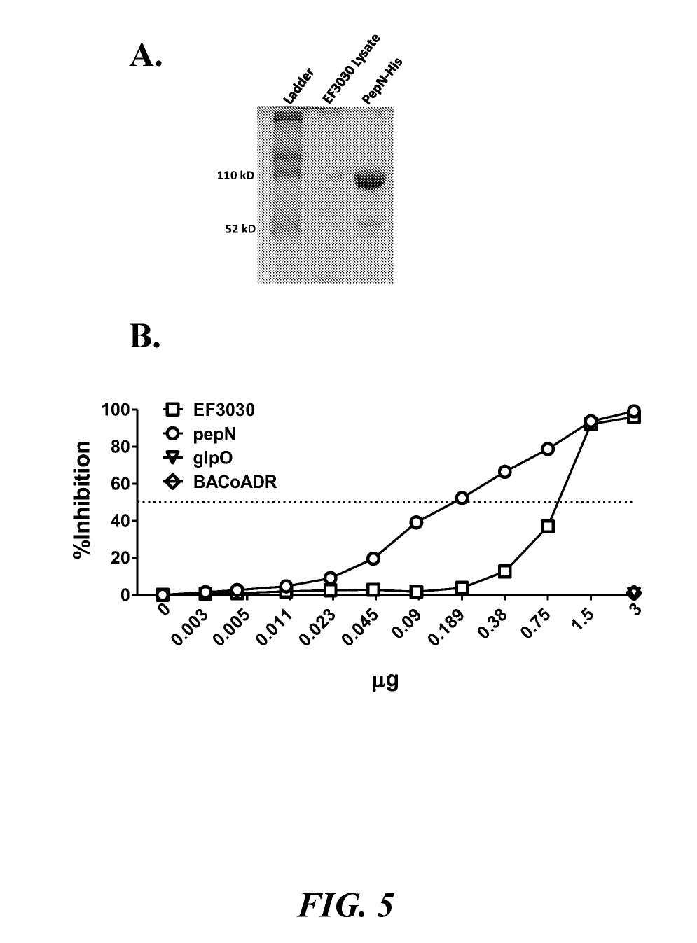

The results described above relied on a clarified lysate which contains a large number of bacterial components. To begin to identify which component was responsible we first assessed a number of basic properties of the inhibitory factor, finding it was heat labile and protease sensitive. Given this we began isolation of the inhibitory factor by fractionation using a series of columns (1) anion exchange column, 2) size exclusion, 3) hydroxyapatite column) with analysis of fractions by a functional assay following each column run. Fractions with inhibitory function obtained by separation on the hydroxyapatite column were resolved by gel electrophoresis. A band with correlative increases in intensity with increasing inhibitory function was excised and sequenced, resulting in identification of Spn aminopeptidase N as a candidate protein. Since the EF3030 genome had not been fully sequenced, and thus its pepN genetic locus was not published, we performed a comparative analysis of the pepN locus in pneumococcal strains TIGR4, D39, AP200, and R6 (GenBank Acc. Nos. AE005672, CP000410, and AE007317, respectively). Our sequencing primers were designed based on the conserved areas between the strains. We have successfully sequenced EF3030 pepN and its surrounding genetic region. PepN was cloned from Spn EF3030 and placed into pTHCm allowing for production of a His-tagged pepN (pepN-His) protein in E. coli. Analysis of lysates from E. coli transduced with pepN versus empty vector showed expression of a band with the expected size of pepN (FIG. 5A). The his tagged protein was subsequently purified by passage over a nickel column. Addition of pepN-His to effector cells resulted in the inhibition of IFNγ production in response to peptide supporting pepN as an inhibitory factor from Spn (FIG. 5B). Addition of his-tagged control proteins B. anthracis coenzyme A-disulfide reductase and a-glycerophosphate oxidase had no effect, demonstrating that the inhibition was due to pepN. No increase in death was associated with pepN-His treatment.

### Example 7

### PepN Results in a Large Reduction in IFNγ mRNA Produced in Response to Peptide Stimulation

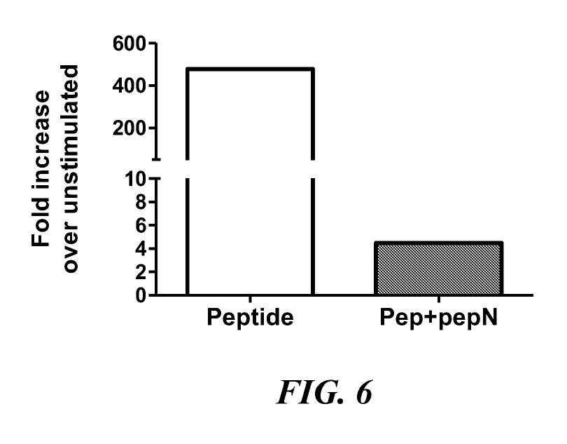

We hypothesized that, similar to treatment with lysate, addition of pepN-His would result in a reduction of IFNγ mRNA. To determine if this were the case, influenza NP-specific effector cells were stimulated with peptide in the presence or absence of pepN-His. mRNA was isolated from effectors and message for IFNγ quantified. Peptide resulted in a 478-fold increase in mRNA Addition of pepN-His reduced this to 4.5-fold (FIG. 6). The level of GAPDH was similar in treated and untreated cells.

### Example 8

### Generation of EF3030 Strains That Overexpress or are Deficient in pepN

While the data described above show that the purified pepN obtained by expression in E. coli inhibited function, the increase in specific activity was lower than would be expected for the purified protein. Without being bound by any particular theory, a number of factors could contribute to this finding: 1) the presence of the His tag could impact activity of the protein; 2) expression in E. coli does not allow glycosylation or other modifications which may be necessary for optimal function; 3) the structure of pepN is relatively unstable and activity is lost during the purification steps; or 4) a co-factor is needed for maximal activity. A significant barrier to address these questions and to further study of this protein is the lack of tools to evaluate the activity of this protein in the context of Spn. To overcome this limitation, we will generate: 1) an EF3030 strain that expresses pepN-His; and 2) a pepN deficient EF3030.

Generation of EF3030 expressing pepN-His: To test the hypothesis that the lower than expected increase in activity of pepN-His was due to improper post-translational modification, we will overexpress pepN in Spn EF3030. We have obtained a non-integrative shuttle vector (vPT-CT7) from Dr. Hui Wu (Wright et al. (2013) PLoS Pathog. 9: e1003274) that will allow for pepN expression in Spn. This vector was designed to have a T7-tag at the C terminus which is useful for rapid immunoaffinity purification. In support of the feasibility of this approach, PepN has been successfully overexpressed in other bacteria, i.e. E. coli (Blevins et al. (2014) J. Immunol. 193: 5076-5087). In addition, creation of this construct will provide a ready source of protein that can be isolated for further in vitro or in vivo study.

To determine whether Spn produced pepN has increased activity, titrated concentrations of pepN-His produced in Spn or E. coli will be added to T effector cells and the ability to inhibit cytokine production determined by ICCS. If optimal pepN activity requires post-translational modifications present when produced in Spn, but not when produced in E. coli, we would expect that the ED₅₀ for Spn produced pepN would be substantially (1-2 logs) lower than that for E. coli produced pepN. If the dose response curves are similar, this would rule out Spn-specific modifications as an explanation for the lower than expected activity of the E. coli produced pepN and instead support the hypothesis that it is either unstable or requires a co-factor, the latter of which is tested below.

Generation of a pepN deficient EF3030: A pepN-deficient (ΔpepN) strain of EF3030 will be generated by replacing the entire pepN gene with an antibiotic marker. Briefly, an artificial PCR construct will be produced that will contain the spectinomycin-resistance gene aad9 linked to ˜1 kb of upstream and downstream flanking regions of the pepN genetic locus. 100 ng of the sequenced construct will be transformed into chemically competent EF3030 and deletion mutants will be screened for spectinomycin-resistance. The entire area will be sequenced to ensure no other unintended genetic changes occurred. We predict this mutant will be viable as pepN has been successfully deleted in other bacteria, e.g. Lactobacillus and E. coli (Kunj i et al. (1996) Mol. Microbiol. 21: 123-131; Chandu et al. (2003) J. Biol. Chem. 278: 5548-5556).

Based on the data described above, we hypothesize that an EF3030 that does not express pepN would be incapable of inhibiting cytokine production in T effector cells. To test this, lysate will be prepared from WT and ΔpepN EF3030. Titrated concentrations of lysate will be added during effector cell stimulation and IFNγ production measured by ICCS. Purified pepN-His will be added with the expectation that it will complement loss of the protein in the deletion mutant. Titrated concentrations of pepN-His alone (i.e. no lysate) will also be utilized. This design will allow us to determine whether a co-factor present in pneumococcus increases activity of pepN. If this is the case we hypothesize that the dose response curve for inhibition is shifted when lysate from ΔpepN EF3030 is present together with pepN-His. While unlikely, it is possible that ΔpepN EF3030 lysate will retain some inhibitory activity. This would suggest an additional factor is present in Spn that on its own has regulatory potential for T cells. This may act together with pepN or independently. Independent factors should show an additive effect when combined, co-factors would be hypothesized to have a synergistic effect.

### Example 9

### Determination of How the Presence of Spn Components Impacts Transcription Factor (TF) Activation in Response to Peptide Stimulation

The data above show that treatment with pepN results in a failure to produce cytokine in response to stimulation. The lack of cytokine production is associated with reduced message. Further, this cannot be overcome by treatment with PMA+ionomycin suggesting a defect in the distal portion of the signaling cascade. As a first step in understanding the mechanistic basis of the regulation by pepN, we will test the hypothesis that pepN-treated cells exhibit differential activation of transcription factors. A large number of TF are thought to be involved in the activation of IFNγ production including NFAT, NFκB, AP-1, ATF2/c-Jun, C/EBP, T-bet, Ets-1, RunX3, STAT1, STAT3, STAT4, and STAT5 (Schoenborn & Wilson (2007) Adv. Immunol. 96: 41-101). There are also a number of repressors that have been identified. These include STAT6, CREB/ATF1, YY1, SMAD3, and GATA-3 (Schoenborn & Wilson (2007) Adv. Immunol. 96: 41-101). Given the large number of potential regulators of IFNγ production, we will employ an approach that allows broad assessment of TF in the nucleus. The Signosis TF Activation 48 factor profiling array allows simultaneous assessment of all but RunX3 and T-bet. These will be measured using the Active Motif TransAm kits. These analyses will be performed using nuclear extracts from peptide stimulated in vitro generated effector cells in the presence or absence of pepN. Nuclear extracts will be isolated at 1 or 3 hours following peptide stimulation. Because in vitro generated effectors are utilized, there are no concerns with cell number or antigen responsiveness for these assays. Thus, we do not expect technical hurdles with the outlined approach.

Potential outcomes and interpretation: The hypothesized outcome from these studies is that the presence of pepN will result in differences in TF activation. Although it is possible that the regulation of mRNA turnover is increased by pepN we think this is less likely given the extremely low level of mRNA detected in cells treated with pepN. Identification of differences in TF patterns in the nucleus may direct us to an arm of the pathway that is dysregulated/inhibited following treatment for pepN. For example, if we see reduced/absent c-jun, it would focus attention on the regulation of MEKK1 or JNK2.

### Example 10

### Determination of How the Presence of Spn pepN Impacts the Activation and Differentiation of Naïve Murine T Cells

The studies described above have shown that the presence of Spn pepN has potent regulatory effects with regard to the production of cytokine by effector cells. It is important to understand whether the differentiation state impacts susceptibility to regulation by Spn pepN. The ability of Spn pepN to negatively affect the activation of naïve T cells could interfere with the activation of T cells in the lung, i.e. in the BALT. The studies described below will evaluate the ability of Spn pepN to inhibit naïve T cell activation and differentiation.

Effect of Spn pepN on T cell activation, proliferation and survival: These studies will test the hypothesis that EF3030 pepN will inhibit the activation and proliferation of naïve CD4⁺ and CD8⁺ T cells. We will use of OT-I (CD8⁺) and OT-II (CD4⁺) TCR transgenic cells as our naïve population. CF SE-labeled OT-I or OT-II cells will be stimulated with LPS-matured bone marrow derived dendritic cells that have been pre-incubated with OVa₂₅₇₋₂₆₄ or OVa₃₂₃₋₃₃₉ peptide, respectively. A high (10⁻⁵M) and low (10⁻⁹M) concentration of each peptide will be used to determine whether increasing TCR signal strength can overcome any negative regulatory effects of Spn pepN. Cultures for OT-II cells will supplemented with the following to promote differentiation into distinct phenotypes: Th1: hIL-2 (10 U/ml)+mIL-12 (10 ng/ml) and neutralizing anti-IL-4 antibody (1 μg/ml); Th2: hIL-2 (25 U/ml) +mIL-4 (10 ng/ml) and neutralizing anti-IL-12 (1 μg/ml)+anti-IFNγ (1 μg/ml) antibody; Th17: hIL-2 (10 U/ml)+mIL-6 (20 ng/ml)+hTGF-β (5 ng/ml) and neutralizing anti-IL-4, anti-IL-12 and anti-IFNγ antibody (all at 1 μg/ml) as we have previously done (Shiner et al. (2014) PLoS One 9: e100175). OT-I cells will be cultured in the presence of IL-2. At 0, 24, 48, or 72 h, cells will be removed from culture and stained with antibodies to CD4 or CD8, CD69, and CD25. The percent of cells positive for CD25 or CD69 as well as the level of expression for each molecule will be quantified. CF SE analysis will allow determination of the percent of cells that entered division as well as the number of divisions that that occurred. Zombie Aqua staining at each time point will be used to quantify cell survival. The timecourse analysis will provide insights into whether the kinetics of activation is altered.

Effect of Spn pepN on T cell acquisition of effector function: To assess cytokine production, cells from the cultures described above (d7 post stimulation) will be stimulated with peptide and DC2.4 cells. DC2.4 cells express both class I and class II to ensure efficient antigen presentation for restimulation of effector function. Titrated amounts of peptide (10⁻⁴M-10⁻¹⁰M) will be employed to determine whether the presence of pepN results in a decrease in functional avidity. GolgiStop or GolgiPlug (BD Biosciences) will be included as recommended by the manufacturer for the particular cytokine measured. Following a five hour stimulation, OT-II cells will stained with anti-CD4 together with antibodies to IFNγ, IL-4, and IL-17. OT-I cells will be stained with anti-CD8 along with antibodies to IFNγ. Anti-CD107 will also be included in these cultures to assess cytotoxicity.

Potential outcomes and interpretation: We hypothesize that naïve T cells will exhibit sensitivity to the inhibitory effects of pepN with regard to proliferation. We propose this based on our hypothesis that pepN interferes with a critical aspect of the membrane distal TCR signaling pathway. As the results from studies described above are generated, we will have a more precise understanding of this regulation. A defect in proliferation would be apparent as a block in entry into cell division or as a decrease in the number of proliferations that occurs. The latter could be associated with a failure to survive past initial division. Analysis of CD25 and CD69 will provide mechanistic insights into changes in proliferation. For example, the failure to upregulate CD25 would suggest an inability to utilize IL-2 may contribute to decreased proliferation. This would lead to future studies aimed at understanding the basis of the failure to upregulate this important participant in the activation and differentiation of T cells. CD69 is among the earliest responses to activating stimuli. The lack of CD69 upregulation would suggest that T cells activation is inhibited at the earliest stage of the activation process.

However, it is possible that naïve T cells are resistant to the effects of Spn pepN. This could occur through differential regulation and/or utilization of transcription factors in naïve and effector cells. For example, the requirement for iKKB in NFkB activation differs between naïve and previously activated cells (for review see Kannan et al. (2012) Int. J. Biochem. Cell Biol. 44: 2129-2134). In addition, accessory signals provided through CD28 or other costimulatory molecules involved in the activation of naïve T cells may overcome/modulate the effects of Spn pepN.

A final possibility is that proliferation will be normal but the presence of Spn pepN selectively inhibits the acquisition of effector function in the cells. A number of transcription factors are involved in the acquisition of effector function and these differ among cell types. T-bet is a critical regulator in the ability to produce IFNγ. mTOR is also important in the acquisition of effector function in these cells. mTORC1 and 2 are both required for the development of Th1 cells. In contrast, Th2 differentiation requires only mTORC2 and Th17 development only mTORC1 (Kannan et al. (2012) Int. J. Biochem. Cell Biol. 44: 2129-2134). Another important difference in naïve versus effector cells is their metabolic state. Effector cells exhibit a shift towards aerobic glycolysis (Maciolek et al. (2014) Curr. Opin. Immunol. 27: 60-74). This may also impact sensitivity to pepN regulation.

We may find that high peptide will overcome the negative effects of Spn pepN. This would be in contrast to what we observe with effectors, as a relatively high amount of soluble peptide (10⁻⁶M) is present during the restimulation to assess function. However, as noted above, the requirements for optimal signaling differ in naïve and activated cells. Such a finding may suggest that the inhibition dampens the signaling cascade as opposed to blocking it.

### Example 11

### Determination of How the Presence of Spn Components Impacts Function in Human T Cells

Thus far our analyses have been performed exclusively in mouse cells. While the effects in this model are impressive, the significance of our finding would be increased by an understanding of the extent to which human cells are susceptible to regulation by Spn pepN. The goal of this aim is to determine whether Spn pepN inhibits function in human CD4⁺ and CD8⁺ effectors as well as its effects on the activation of human naïve CD4⁺ and CD8⁺ T cells.

Are human effector cells susceptible to negative regulation by Spn pepN? Blood will be collected from normal human donors in the clinical research unit (CRU) at Wake Forest School of Medicine. Peripheral blood mononuclear cells will be isolated using Lymphoprep. CD8⁺ and CD4⁺ T cells will be purified from mononuclear cells with the CD8⁺ and CD4⁺ T cell isolation kit from Miltenyi Biotec. These kits result in populations that are >95% pure. Cells will be cultured with 10 μg/ml immobilized anti-CD3 antibody along with 10 μg/ml anti-CD28 antibody or with phorbol myristate acetate and ionomycin. Cells will be cultured for 5 days to generate an effector population. CD4⁺ T cells from each donor will be differentiated into Th1, Th2 and Th17 effector cells by stimulation in the presence of differentiating cytokines (Acosta-Rodriguez et al. (2007) Nat. Immunol. 8: 942-949; Cousins et al. (2002) J. Immunol. 169: 2498-2506. On d5, cells will restimulated with PMA+ion in the presence of titrated concentrations of Spn [pepN] and the production of IFNγ, TNFα, IL-4, IL-5, and IL-17 determined by flow cytometry.

Does the presence of Spn pepN negatively impact the proliferation and differentiation of naïve human CD4⁺ and CD8⁺ T cells? Naïve CD4⁺ and CD8⁺ T cells from healthy donors will be isolated using Miltenyi Biotec bead selection kits as per the manufacturer's instructions. Isolated populations will be labeled with CFSE and cultured with 10 μg/ml immobilized anti-CD3 antibody along with 10 μg/ml anti-CD28 antibody or with phorbol PMA+ionomycin. Titrated concentrations of Spn pepN will be added to the cultures. On days 1-5 post stimulation, cells will be harvested, counted and stained with antibodies to CD4 or CD8 along with CD69 and CD25. Marker expression level and CFSE dilution profile will be determined by flow cytometry. At each harvest point, cells will also be stained with Zombie Dye to measure viability.

Potential outcomes and interpretation: The above experiments will determine the susceptibility of human CD4⁺ and CD8⁺ T cells to inhibition by Spn. Further potential changes as a result of differentiation will also be determined. We hypothesize that there will be similarities in the response of mouse and human T cells, given the similarities in TCR signaling and activation requirements. However, it is always possible that a species specific difference in a molecule involved in the function of pepN differs in mouse and humans that prevents the inhibitory activity. If this is the case, our future plan is to test T cells from other species where Spn causes disease, e.g. pigs and cattle.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.

Although the foregoing subject matter has been described in some detail by way of illustration and example for purposes of clarity of understanding, it will be understood by those skilled in the art that certain changes and modifications can be practiced within the scope of the appended claims.

1. An isolated or recombinant aminopeptidase N (pepN) polypeptide, wherein the isolated or recombinant pepN polypeptide comprises the amino acid sequence of SEQ ID NO:1 or a functional variant thereof, wherein the functional variant thereof has inflammatory cytokine production and cytolysis inhibiting activity and comprises an amino acid sequence at least 80% identical to the amino acid sequence of SEQ ID NO:1.

2. The isolated or recombinant pepN polypeptide of claim 1, wherein the isolated or recombinant pepN polypeptide is fused to a heterologous polypeptide.

3. The isolated or recombinant pepN polypeptide of claim 2, wherein the heterologous polypeptide is an epitope tag.

4. The isolated or recombinant pepN polypeptide of claim 1, wherein the functional variant is a functional fragment of an amino acid sequence at least 80% identical to the amino acid sequence of SEQ ID NO: 1.

5. A composition comprising the isolated or recombinant pepN polypeptide of claim 1 in a pharmaceutically acceptable carrier.

6. A method of inhibiting inflammatory cytokine production and cytolysis in a subject in need thereof, the method comprising administering to the subject an inflammatory cytokine production and cytolysis inhibiting amount of an isolated or recombinant aminopeptidase N (pepN) polypeptide, wherein the isolated or recombinant pepN polypeptide comprises the amino acid sequence of SEQ ID NO:1 or a functional variant thereof, wherein the functional variant thereof has inflammatory cytokine production and cytolysis inhibiting activity and comprises an amino acid sequence at least 80% identical to the amino acid sequence of SEQ ID NO: 1.

7. The method of claim 6, wherein the isolated or recombinant pepN polypeptide is fused to a heterologous polypeptide.

8. The method of claim 7, wherein the heterologous polypeptide is an epitope tag.

9. The method of claim 6, wherein the functional variant is a functional fragment of an amino acid sequence at least 80% identical to the amino acid sequence of SEQ ID NO: 1.

10. The method of claim 6, wherein the isolated or recombinant pepN polypeptide is in a pharmaceutically acceptable carrier.

11. The method of claim 6, wherein the inflammatory cytokine is selected from the group consisting of Interleukin 1 (IL-1), Interleukin 2 (IL-2), Interleukin 5 (IL-5), Interleukin 6 (IL-6), Interleukin 8 (IL-8), Tumor Necrosis Factor alpha (TNFa.), and Interferon gamma (IFNy).

12. The method of claim 6, wherein the subject is a human.

13-21. (canceled)

22. A method of treating pneumococcal infection in a subject in need thereof, the method comprising administering to the subject a therapeutically effective amount of an anti aminopeptidase N (pepN) polypeptide inhibitory agent, wherein the anti-pepN polypeptide inhibitory agent is an anti-pepN polypeptide antibody or antigen-binding fragment thereof, a small molecule pepN polypeptide inhibitor, an RNA or DNA aptamer that binds or physically interacts with a pepN polypeptide, a soluble pepN polypeptide receptor, a pepN polypeptide specific antisense molecule, or a pepN polypeptide specific siRNA molecule.

23. The method of claim 22, wherein the pepN polypeptide is an isolated or recombinant pepN polypeptide comprising the amino acid sequence of SEQ ID NO:1 or a functional variant thereof, wherein the functional variant thereof has inflammatory cytokine production and cytolysis inhibiting activity and comprises an amino acid sequence at least 80% identical to the amino acid sequence of SEQ ID NO:1.

24. The method of claim 23, wherein the functional variant is a functional fragment of an amino acid sequence at least 80% identical to the amino acid sequence of SEQ ID NO:1.

25. The method of claim 22, wherein the subject is a human.


SEQUENCE LISTING (1 sequence)

SEQ ID NO: 1 - protein, 848 aa, Streptococcus pneumoniae
MQAVEHFIKQFVPEHYDLFLDLSRETKTFSGKVTITGQAQSDRISLHQKDLEITSVEVAGQARPFTVDHDNEALHIELAEAGQVELVLAFSGKITDNMTGIYPSYYTVDGVKKEVLSTQFESHFAREAFPCVDEPEAKATFDLALRFDQAAGELALSNMPEIDVDNRKETGIWKFETTPRMSSYLLAFVAGDLQGVTAKTKNGALVGVYSTKAHPLSNLDFSLDIAVRSIEFYEDYYGVKYPIPQSLHIALPDFSAGAMENWGLVTYREVYLVVDENSTFASRQQVALVVAAALAAQWFGNLVTMKWWDDLWLNESFANMMEYVCVDTIEPSWNIFEDFQTGGVPLALERDATDGVQSVHVEVKHPDEINTLFDGAIVYAKGSRLMHMLRRWLGDADFAKGLHAYFEKHQYSNTIGSDLWDALGQASGRDVAAFMDSWLEQPGYPVLTVKVENDVLKISQKQFFIGENEDKNRLWVVPLNSNWKGLPDTLETESIEIPGYAALLAENEGALRLNTENTAHYITDYQGDLLEAVLAELETLDNTSKLQIVQERRLLAEAGHISYADLLPVLDKLAKEESYLVVSAVSQVISALERFIDEGTDAETAFKGLVAKLARHNYDRLGFEAKDGESDEDELVRQLAVSMMIRSNDAEASQVASQIFATHKENLAGLPAAIRSQVLINEMKHHETKDLLALYLDTYTHATDAVFKRQLAAALAYSTDADNIQNLITSWKDKFVVKPQDLSAWYYQFLAHQATQKTAWSWARENWAWIKAALGGDMSFDSFVILPAHVFKTQQRLAEYKEFFEPQLSDLALSRNIGMGIKEIAARVDLISREKAAVEAVVLQYGNA